(19) United States
(12) Patent Application Publication
(10) Pub. No.: US 2020/0294288 A1
(43) Pub. Date: Sep. 17, 2020

(54) SYSTEMS AND METHODS OF COMPUTED TOMOGRAPHY IMAGE RECONSTRUCTION

(71) Applicant: The UAB Research Foundation, Birmingham, AL (US)

(72) Inventor: Andrew Dennis Smith, Hoover, AL (US)

(21) Appl. No.: 16/817,602

(22) Filed: Mar. 12, 2020

Related U.S. Application Data
(60) Provisional application No. 62/818,085, filed on Mar. 13, 2019.

(51) Int. Cl. G06T 11/00; G06T 5/00; G06T 7/00; G06N 3/08; G16H 50/20; A61B 6/03; A61B 6/00

(52) U.S. Cl. CPC G06T 11/008; G06T 5/002; G06T 7/0012; G06N 3/08; G16H 50/20; A61B 6/032; G06T 2207/10081; A61B 6/504; G06T 2211/408; G06T 2210/41; G06T 2207/20084; G06T 2207/20081; A61B 6/5223

(57) ABSTRACT

Methods for reconstructing an image can include, inter alia, (i) reconstructing a contrast-enhanced output CT image from a nonenhanced input CT image, (ii) reconstructing a nonenhanced output CT image from a contrast-enhanced CT image, (iii) reconstructing a dual-energy, contrast-enhanced output CT image from a single-energy, contrast-enhanced CT image, and/or (iv) reconstructing a full-dose, contrast-enhanced CT image from a low-dose, contrast-enhanced CT image.

### CROSS-REFERENCE TO RELATED APPLICATIONS

This application claims priority to and the benefit of U.S. Provisional Patent Application Ser. No. 62/818,085, filed Mar. 13, 2019 and titled “SYSTEMS AND METHODS OF COMPUTED TOMOGRAPHY IMAGE RECONSTRUCTION,” which is incorporated herein by this reference in its entirety.

### BACKGROUND

### Technical Field

This disclosure generally relates to image processing. More specifically, the present disclosure relates to reconstruction of medical image data.

### Related Technology

Image reconstruction in the image processing field can, at a basic level, represent the cancellation of noise components from images using, for example, an algorithm or image processing system or by estimating lost information in a low-resolution image to reconstruct a high-resolution image. While simplistic in conception, image reconstruction is notoriously difficult to implement—despite what Hollywood spy movies would suggest from the frequent use of on-demand, instant high-resolution enhancement of satellite images by simply windowing the desired area to be enhanced.

Noise can be acquired or compounded at various stages, including, during image acquisition or any of the pre- or post-processing steps. Local noise typically follows a Gaussian or Poisson distribution, whereas other artifacts, like streaking, are typically associated with non-local noise. Denoising filters, such as a Gaussian smoothing filter or patch-based collaborative filtering, can be helpful in some circumstances to reduce the local noise within an image. However, there are limited methods available for dealing with non-local noise, and this tends to make the conversion of images from low resolution to high resolution, or to otherwise reconstruct the images, both difficult and time consuming.

Additionally, image contrast is a critical component of medical imaging. Low contrast medical images make it difficult to differentiate normal structures from abnormal structures. In the case of computed tomography (CT) and magnetic resonance imaging (MRI), one method to improve contrast on an image is to deliver a contrast agent to the patient. Contrast agents can be delivered intravenously, intra-arterially, percutaneously, or via an orifice (e.g., oral, rectal, urethral, etc.). The purpose of the contrast agent is to improve image contrast and thereby improve diagnostic accuracy. Unfortunately, some patients respond adversely to contrast agents. Even if tolerated, however, contrast agents are often associated with multiple side effects, and the amount of contrast that can be delivered to a patient is finite. As such, there is a limit on contrast improvement of medical image data using known contrast agents and known image acquisition and post-processing techniques.

Accordingly, there are a number of disadvantages with image reconstruction that can be addressed.

### BRIEF SUMMARY

Embodiments of the present disclosure solve one or more of the foregoing or other problems in the art with image reconstruction, especially the reconstruction of CT images. An exemplary method includes reconstructing an output image from an input image using a deep learning algorithm such as a convolutional neural network that can be wholly or partially unsupervised/supervised.

In some embodiments, the images are CT images, and methods of the present disclosure include, inter alia, (i) reconstructing a contrast-enhanced output CT image from a nonenhanced input CT image, (ii) reconstructing a nonenhanced output CT image from a contrast-enhanced CT image, (iii) reconstructing a high-contrast output CT image from a contrast-enhanced CT image obtained with a low dose of a contrast agent, and/or (iv) reconstructing a low-noise, high-contrast CT image from a CT obtained with low radiation dose and a low dose of a contrast agent.

An exemplary method for reconstructing a computed tomography image includes receiving an input CT image and reconstructing an output CT image from the input CT image using an image reconstruction algorithm generated from a supervised convolutional neural network having one or more parameters of one or more layers of the supervised convolutional neural network informed by received user input.

In one aspect, the input CT image is a nonenhanced CT image and reconstructing the output CT image comprises reconstructing a virtual contrast-enhanced CT image from the nonenhanced CT image. In one aspect, the method can further comprise training the convolutional neural network using a set of images that comprises a plurality of paired multiphasic CT images, wherein each of the paired multiphasic images comprises a nonenhanced CT image and a contrast-enhanced CT image of substantially a same slice from a same patient.

In one aspect, the input CT image is a contrast-enhanced CT image and reconstructing the output CT image comprises reconstructing a virtual nonenhanced CT image from the contrast-enhanced CT image. In one aspect, the method can further comprise training the convolutional neural network using a set of images that comprises a plurality of paired multiphasic CT images, wherein each of the paired multiphasic images comprises a nonenhanced CT image and a contrast-enhanced CT image of substantially a same slice from a same patient.

In one aspect, the input CT image is a single-energy, contrast-enhanced or unenhanced CT image and reconstructing the output CT image comprises reconstructing a virtual dual-energy, contrast-enhanced CT image from the single-energy, contrast-enhanced or unenhanced CT image. In one aspect, the method further comprises training the convolutional neural network using a training set comprising a plurality of dual-energy contrast-enhanced CT images, wherein for each dual-energy, contrast-enhanced CT image within the training set, a 70 keV portion of an associated dual-energy, contrast-enhanced CT image is used as a training input CT image and the associated dual-energy, contrast-enhanced CT image is used as a training output CT image.

In one aspect, the input CT image is a low-dose, contrast-enhanced CT image.

In one aspect, the low-dose, contrast-enhanced CT image is obtained from a patient having received a contrast dosage calculated to be at least 10% less than a full-dose of contrast.

In one aspect, the low-dose, contrast-enhanced CT image is obtained from a patient having received a contrast dosage calculated to be between about 10-20% of a full-dose of contrast.

In one aspect, the low-dose, contrast-enhanced CT image is obtained from a patient having received a contrast dosage calculated to be at least 10%, preferably at least about 20%, more preferably at least about 33% less than a full-dose of contrast.

In one aspect, the contrast is intravenous iodinated contrast. In one aspect, reconstructing the output image comprises reconstructing a virtual full-dose, contrast-enhanced CT image from the low-dose, contrast-enhanced CT image, the virtual full-dose, contrast-enhanced CT image being reconstructed without sacrificing image quality or accuracy. In one aspect, the method further comprises training the convolutional neural network using a training set of paired low-dose, contrast-enhanced and full-dose, contrast-enhanced CT images, wherein for each pair of low-dose, contrast-enhanced and full-dose, contrast-enhanced CT images within the training set, the low-dose, contrast-enhanced CT image is used as a training input CT image and the associated full-dose, contrast-enhanced CT image is used as a training output CT image.

In one aspect, the method further comprises reducing a likelihood of contrast-induced nephropathy or allergic-like reactions in a patient undergoing contrast-enhanced CT imaging, wherein reducing the likelihood of contrast-induced nephropathy or allergic-like reactions in the patient comprises administering the low dose of contrast to the patient prior to or during CT imaging.

Embodiments of the present disclosure additionally include various computer program products having stored thereon computer-executable instructions that, when executed by one or more processors of a computer system, cause the computer system to reconstruct a CT image.

In one aspect, the computer system reconstructs virtual contrast-enhanced CT images from a patient undergoing nonenhanced CT imaging by performing a method that includes receiving an input CT image and reconstructing an output CT image from the input CT image using an image reconstruction algorithm generated from a supervised convolutional neural network having one or more parameters of one or more layers of the supervised convolutional neural network informed by received user input. In such a method, the input CT image is a nonenhanced CT image and reconstructing the output CT image comprises reconstructing a virtual contrast-enhanced CT image from the nonenhanced CT image. The method further includes training the convolutional neural network using a set of images that comprises a plurality of paired multiphasic CT images, wherein each of the paired multiphasic images comprises a nonenhanced CT image and a contrast-enhanced CT image of substantially a same slice from a same patient.

In one aspect, the computer system reconstructs nonenhanced CT image data from a patient undergoing contrast-enhanced CT imaging by performing a method that includes receiving an input CT image and reconstructing an output CT image from the input CT image using an image reconstruction algorithm generated from a supervised convolutional neural network having one or more parameters of one or more layers of the supervised convolutional neural network informed by received user input. In such a method, the input CT image is a contrast-enhanced CT image and reconstructing the output CT image comprises reconstructing a virtual nonenhanced CT image from the contrast-enhanced CT image. The method additionally includes training the convolutional neural network using a set of images that comprises a plurality of paired multiphasic CT images, wherein each of the paired multiphasic images comprises a nonenhanced CT image and a contrast-enhanced CT image of substantially a same slice from a same patient.

In one aspect, the computer system reconstructs dual-energy, contrast-enhanced CT image data from a patient undergoing single-energy, contrast-enhanced or nonenhanced CT imaging by performing a method that includes receiving an input CT image and reconstructing an output CT image from the input CT image using an image reconstruction algorithm generated from a supervised convolutional neural network having one or more parameters of one or more layers of the supervised convolutional neural network informed by received user input. In such a method, the input CT image is a single-energy, contrast-enhanced or unenhanced CT image and reconstructing the output CT image comprises reconstructing a virtual dual-energy, contrast-enhanced CT image from the single-energy, contrast-enhanced or unenhanced CT image. The method additionally includes training the convolutional neural network using a training set comprising a plurality of dual-energy contrast-enhanced CT images, wherein for each dual-energy, contrast-enhanced CT image within the training set, a 70 keV portion of an associated dual-energy, contrast-enhanced CT image is used as a training input CT image and the associated dual-energy, contrast-enhanced CT image is used as a training output CT image.

Embodiments of the present disclosure additionally include computer systems for reconstructing an image. An exemplary computer system includes one or more processors and one or more hardware storage devices having stored thereon computer-executable instructions, when executed by the one or more processors, cause the computer system to at least (i) receive a low-dose, contrast-enhanced computed tomography (CT) image captured from a patient who received a dosage of intravenous iodinated contrast calculated to be at least 10% less than a full-dose of intravenous iodinated contrast; and (ii) reconstruct an output CT image from the low-dose, contrast-enhanced CT image using an image reconstruction algorithm generated from a convolutional neural network. In one aspect, the output CT image is a virtual full-dose, contrast-enhanced CT image. In one aspect, the convolutional neural network is trained using a training set of paired low-dose, contrast-enhanced and full-dose, contrast-enhanced CT images such that for each pair of low-dose, contrast-enhanced and full-dose, contrast-enhanced CT images within the training set, the low-dose, contrast-enhanced CT image is used as a training input CT image and the associated full-dose, contrast-enhanced CT image is used as a training output CT image.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates an exemplary schematic of a computing environment for facilitating machine learning techniques to reconstruct CT images.

FIG. 2 illustrates an exemplary schematic that provides additional detail for the diagnostics component of FIG. 1.

FIG. 3 illustrates a flow chart of an exemplary method of the present disclosure.

FIG. 4 illustrates another flow chart of an exemplary method of the present disclosure.

FIG. 5 illustrates yet another flow chart of an exemplary method of the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims.

### Overview of Computed Tomography (CT)

Computed tomography is a medical imaging technique that uses X-rays to image fine slices of a patient's body, thereby providing a window to the inside of a patient's body without invasive surgery. Radiologists use CT imaging to evaluate, diagnose, and/or treat any of the myriad internal maladies and dysfunctions. The majority of CT scanners Most CT is performed using single energy CT scanners. The most common type of CT imaging of the abdomen is performed with concomitant administration of intravenous iodinated (IV) contrast to the patient. It is currently not possible to accurately evaluate for fatty liver disease or to fully characterize various masses (e.g., adrenal, renal, liver, etc.) on single-energy contrast-enhanced CT images. Artificial Intelligence (AI) can be used reconstruct CT images, and it is possible to train an AI algorithm to convert single-energy contrast-enhanced CT images into virtual enhanced images. The virtual enhanced images could be used to quantify liver fat to diagnose fatty liver disease and would also be helpful for characterization of various masses.

### Overview of the Disclosed Embodiments

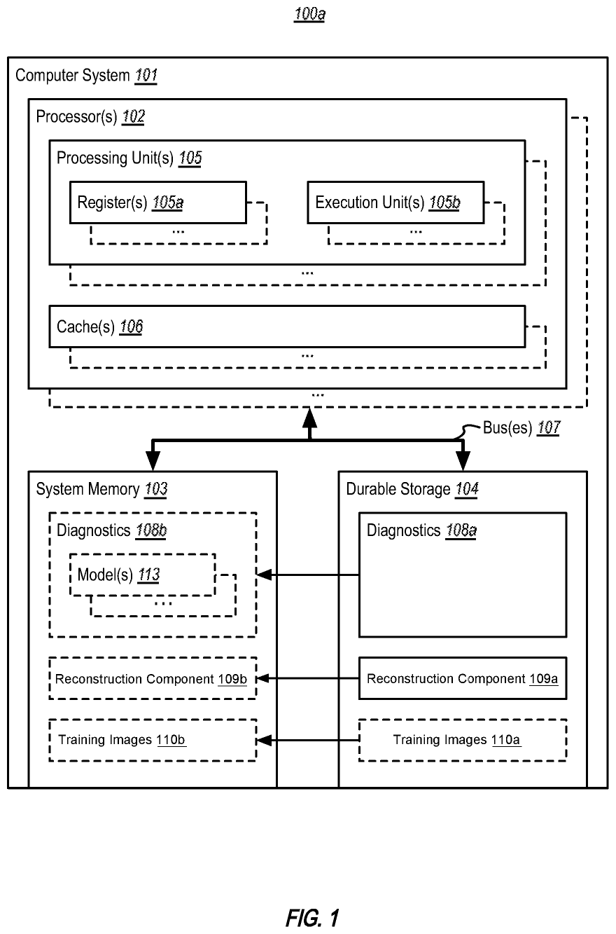

Embodiments of the present disclosure utilize training sets of CT images to train deep learning algorithms, such as convolutional neural nets (or similar machine learning techniques), and thereby enable the reconstruction of CT image data. For example, FIG. 1 illustrates an example computing environment 100a that facilitates use of machine learning techniques to automatically identify reconstruction paradigms that, when applied to an input image, reconstruct input images as any of a virtual contrast-enhanced CT image, virtual unenhanced CT image, virtual dual-energy, contrast-enhanced CT image, and/or virtual full-dose, contrast-enhanced CT image. By so doing, additional clinical information can be gleaned from the data on hand without necessarily having to perform another scan. Embodiments of the present disclosure enable this to occur rapidly and thereby provide the radiologist or other healthcare professional with potentially relevant clinical information that can positively impact patient care.

As depicted in FIG. 1, a computing environment 100a can utilize a special-purpose or general-purpose computer system 101 that includes computer hardware, such as, for example, one or more processors 102, system memory 103, and durable storage 104, which are communicatively coupled using one or more communications buses 107.

As shown, each processor 102 can include (among other things) one or more processing units 105 (e.g., processor cores) and one or more caches 106. Each processing unit 105 loads and executes computer-executable instructions via the caches 106. During execution of these computer-executable instructions at one more execution units 105b, the instructions can use internal processor registers 105a as temporary storage locations and can read and write to various locations in system memory 103 via the caches 106. In general, the caches 106 temporarily cache portions of system memory 103; for example, caches 106 might include a “code” portion that caches portions of system memory 103 storing application code, and a “data” portion that caches portions of system memory 103 storing application runtime data. If a processing unit 105 requires data (e.g., code or application runtime data) not already stored in the caches 106, then the processing unit 105 can initiate a “cache miss,” causing the needed data to be fetched from system memory 103—while potentially “evicting” some other data from the caches 106 back to system memory 103.

As illustrated, the durable storage 104 can store computer-executable instructions and/or data structures representing executable software components. Correspondingly, during execution of this executable software at the processor(s) 102, one or more portions of the executable software can be loaded into system memory 103. For example, the durable storage 104 is shown as potentially having stored thereon code and/or data corresponding to a diagnostics component 108a, a reconstruction component 109a, and a set of input/output training images 110a. Correspondingly, system memory 103 is shown as potentially having resident corresponding portions of code and/or data (i.e., shown as diagnostics component 108b, reconstruction component 109b, and the set of training images 110b). As also shown, durable storage 104 can also store data files, such as a plurality of parameters associated with machine learning techniques, parameters or equations corresponding to one or more layers of a convolutional neural net, or similar—all, or part, of which can also be resident in system memory 103, shown as a plurality of output images 112b.

In general, the diagnostics component 108 utilizes machine learning techniques to automatically identify differences between a plurality of input and output images within the training set. In doing so, the machine learning algorithm can generate a reconstruction paradigm by which a new input image can be reconstructed into the desired output (e.g., a nonenhanced CT image reconstructed as a contrast-enhanced CT image or other examples as disclosed herein) with a high enough fidelity and accuracy that a physician, preferably a radiologist, can gather actionable information from the image. In some instances, the actionable information is evidence that a follow-up contrast-enhanced CT scan should be performed on the patient. In other instances, the actionable information may be an indication that a follow-up contrast-enhanced CT scan is unnecessary. In some embodiments, the actionable information identifies or confirms a physician's diagnosis of a malady or dysfunction.

The actionable information can, in some instances, provide the requisite information for physicians to timely act for the benefit of the patient's health. Embodiments of the present disclosure are generally beneficial to the patient because it can decrease the total amount of radiation and/or the number of times the patient is exposed to radiation. It can also beneficially free up time, personnel, and resources for other procedures if the follow-up CT scan was determined to be unnecessary. For instance, the radiology technician, the radiologist, or other physicians or healthcare professionals, in addition to the CT scanner, will not be occupied with performing a follow-up contrast-enhanced CT scan, and those resources can be utilized to help other patients. Accordingly, embodiments of the present disclosure may additionally streamline the physician's workflow and allow the physician to do more work in less time—and in some embodiments while spending less money on equipment and/or consumables (e.g., contrast agent). Embodiments of the present disclosure, therefore, have the potential to make clinics and hospitals more efficient and more responsive to patient needs.

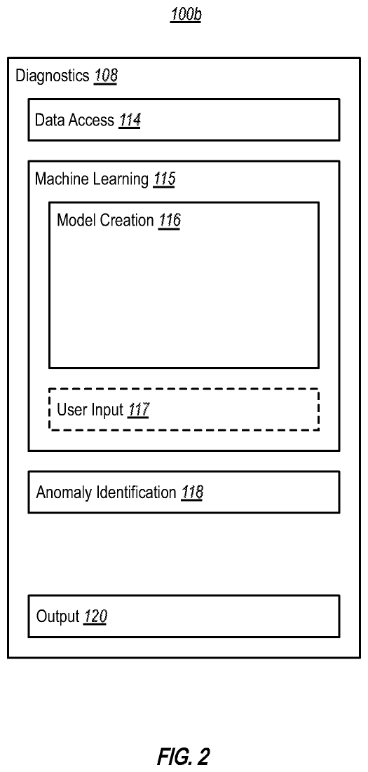

Referring back to the Figures, FIG. 2 illustrates an exemplary system 100b that provides additional detail of the diagnostics component 108 discussed above and illustrated in FIG. 1. As depicted in FIG. 2, the diagnostics component 108 can include a variety of components (e.g., data access 114, machine learning 115, anomaly identification 118, output 120, etc.) that represent various functionality the diagnostics component 108 might implement in accordance with various embodiments described herein. It will be appreciated that the depicted components—including their identity and arrangement—are presented merely as an aid in describing various embodiments of the diagnostics component 108 described herein and that these components are non-limiting to how software and/or hardware might implement various embodiments of the diagnostics component 108 described herein, or of the particular functionality thereof.

The machine learning component 115 applies machine learning techniques to the plurality of images within the training set. In some embodiments, these machine learning techniques operate to identify whether specific reconstructions or reconstruction parameters appear to be normal (e.g., typical or frequent) or abnormal (e.g., atypical or rare). Based on this analysis, the machine learning component 115 can also identify whether specific output images 112 appear to correspond to normal or abnormal output images 112. It is noted that use of the terms “normal” and “abnormal” herein does not necessarily imply whether the corresponding output image is visually pleasing or distorted, or that one image is good or bad, correct or incorrect, etc.—only that it appears to be an outlier compared to similar data points or parameters seen across the output images in the training set.

While the machine learning component 115 could use a variety of machine learning techniques, in some embodiments the machine learning component 115 develops one or more models over the training set, each of which captures and characterizes different attributes obtained from the output images and/or reconstructions. For example, in FIG. 2, the machine learning component 115 includes a model creation component 116 that creates one or more models 113 (shown in FIG. 1) over the training set, one of which may be a component of or wholly encompassing a convolutional neural net. In one embodiment, the convolutional neural net can be unsupervised, taking the training set and determining a reconstruction paradigm without user interaction or guidance related to layer parameters, “normal” or predicted reconstructions, “abnormal” or unpredicted reconstructions, or the like.

In other embodiments, the convolutional neural network can be partially supervised. As shown in FIG. 2, the machine learning component 115 might include a user input component 117. As such, the machine learning component 115 can utilize user input when applying its machine learning techniques. For example, the machine learning component 115 might utilize user input specifying particular parameters for components within layers of the neural net or might utilize user input to validate or override a classification or defined parameter. Similarly, the user input 117 can be used by the machine learning component 115 to validate which images generated from a given reconstruction paradigm were accurate.

For example, a training set may include non-contrast and contrast CT images from the same set of patients, and the machine learning component 115 can be tasked with developing a reconstruction paradigm that reconstructs a high contrast CT image from a non-contrast CT image. A subset of the training set can be used to train the convolutional neural net, and the resulting reconstruction paradigm can be validated by inputting non-contrast CT images from a second subset of the training set, applying the image reconstruction paradigm generated by the machine learning component, which generates a respective output image in the form of a (predicted) contrast CT image, and receiving user input that indicates whether the output image is a normal or abnormal image. The user input can be based, for example, on a comparison of the corresponding contrast CT image in the second subset of the training set that corresponds to the non-contrast CT image input into the machine learning component. Thus, the machine learning component 115 can utilize supervised machine learning techniques, in addition or as an alternative to unsupervised machine learning techniques.

In some embodiments, the convolutional neural network may be formed by stacking different layers that collectively transform input data into output data. For example, the input image obtained through CT scanning is processed to obtain a reconstructed CT image by passing through a plurality of convolutional layers, for example, a first convolutional layer, a second convolutional layer, . . . , an (n+1)^{th} convolutional layer, where n is a natural number. These convolutional layers are essential blocks of the convolutional neural network and can be arranged serially or in clusters. In one general, though exemplary, arrangement, an input image is followed by a number of “hidden layers” within convolutional neural network, which usually includes a series of convolution and pooling operations extracting feature maps and performing feature aggregation, respectively. These hidden layers are then followed by fully connected layers providing high-level reasoning before an output layer produces predictions (e.g., as an output image).

Each layer of a convolutional neural network can have parameters that consist of, for example, a set of learnable convolutional kernels, each of which has a certain receptive field and extends over the entire depth of the input data. In a forward process, each convolutional kernel is convolved along a width and a height of the input data, a dot product of elements of the convolutional kernel and the input data is calculated, and a two-dimensional activation map of the convolutional kernel is generated. As a result, the network may learn a convolutional kernel which can be activated only when a specific type of characteristic is seen at a certain input spatial position. Activation maps of all the convolutional kernels can be stacked in a depth direction to form all the output data of the convolutional layer. Therefore, each element in the output data may be interpreted as an output of a convolutional kernel which sees a small area in the input and shares parameters with other convolutional kernels in the same activation map.

It should be appreciated that other deep learning models may be used, as appropriate, such as deep autoencoders and generative adversarial networks. These foregoing deep learning models may be advantageous for embodiments relying on unsupervised learning tasks.

Returning to the Figures, the output component 120 synthesizes, if necessary, output data from the deep learning algorithm and outputs reconstructed output images. The output component 120 could output to a user interface (e.g., corresponding to diagnostics component 108) or to some other hardware or software component (e.g., persistent memory for later recall and/or viewing). If the output component 120 outputs to a user interface, this user interface could visualize one or more similar reconstructed images for comparison. If the output component 120 outputs to another hardware/software component, that component might act on that data in some way. For example, the output component could output a reconstructed image that is then further acted upon by a secondary machine learning algorithm to, for example, smooth or denoise the reconstructed image.

### Exemplary Embodiments of the Present Disclosure

Most CT imaging is performed using single energy CT scanners. The most common type of CT imaging of the abdomen is performed with intravenous iodinated contrast. It is currently not possible to accurately evaluate for fatty liver disease or to fully characterize various masses (e.g., adrenal, renal, liver, etc.) on single-energy contrast-enhanced CT images. Instead, dual-energy, contrast-enhanced CT scanners are typically used to acquire the requisite definition to evaluate fatty liver disease or to fully characterize various masses. Unfortunately, dual-energy, contrast-enhanced CT scanners are not prevalent in patient care facilities (e.g., many hospitals) and are typically associated with specialized medical imaging centers. These types of scanners are also prohibitively expensive to operate. It is often not practical for a patient to travel to a distant facility for the dual-energy, contrast-enhanced CT scan, but even if the patient was able to travel to—and pay for—the dual-energy contrast-enhanced CT scan, the patient is once again being subjected to radiation.

Further, the most common type of CT imaging of the abdomen is performed with intravenous iodinated contrast. Iodinated contrast is useful for improving CT image contrast but is associated with risks including contrast-induced nephropathy and allergic-like reactions, including anaphylactic reactions. Current efforts are underway to limit the dose of iodinated contrast but doing so causes the signal to noise ratio to break down, resulting in unclear images. Most denoising filters have reached the limit of their utility in this respect such that a lower limit has been reached with respect to balancing patient health (i.e., lower iodinated contrast levels) with image quality. Further complicating matters, current image reconstruction algorithms are not capable of improving contrast in a manner that matches the normal biodistribution and pattern seen in normal and pathologic states.

Embodiments of the present disclosure employ deep learning algorithms, such as those discussed above, to improve the diagnostic accuracy of routine single energy CT or dual energy CT in a variety of settings. The vast majority of CT scanners in the world are single energy CT scanners, and embodiments of the present disclosure can use the CT images produced by these scanners to generate virtual single-energy contrast-enhanced CT images without subjecting the patient to any iodinated contrast. Nonenhanced CT images can be reconstructed into virtual single-energy contrast-enhanced CT images by, for example, training a deep learning algorithm (e.g., a convolutional neural network) with a large number (e.g., 100,000) of de-identified multiphasic (i.e., unenhanced and contrast-enhanced) CT studies. Using this dataset, paired multiphasic images made up of a nonenhanced CT image and a corresponding contrast-enhanced CT image are used as a training input CT image and training output CT image, respectively. The deep learning algorithm can be trained in an unsupervised or supervised manner, and because the multiphasic dataset is agnostic to whether a virtual contrast-enhanced image is reconstructed from an input nonenhanced CT image versus a virtual nonenhanced CT image being reconstructed from an input contrast-enhanced CT image, the deep learning algorithm can be trained to generate a virtual contrast-enhanced CT image from an input nonenhanced CT image or to generate a virtual nonenhanced CT image form an input contrast-enhanced CT image.

Generating a virtual contrast-enhanced CT image from a nonenhanced CT image input can expand the utility of nonenhanced CT imaging and decrease the costs associated therewith. Further, patients avoid intravenous contrast and the potential complications associated therewith.

Further embodiments of the present disclosure provide methods for reconstructing a virtual dual-energy (or high contrast image), contrast-enhanced CT output image from a single-energy, contrast-enhanced CT input image. Similar to that described above, a deep learning algorithm can be trained on a dataset that includes a large number of de-identified dual-energy CT studies (e.g., 10,000 studies). For example, a convolutional neural network can be trained to convert the 70 keV portion of each dual-energy CT image (equivalent to the single energy CT image) into the corresponding virtual dual-energy CT image in the study set. That is, the data using the real dual-energy CT images acts as the training set and reference standard. The resulting trained convolutional neural network can be operable to reconstruct a single-energy CT image into a virtual dual-energy CT image. Doing so can substantially increase the utility of single-energy CT scanners and make dual-energy CT image equivalents more widely available to patients, which, in turn, can lead to an increase in patient care.

As another example, embodiments of the present disclosure can enable a reduction in the amount and/or concentration of iodinated contrast administered to the patient without sacrificing image quality and/or accuracy. In some instances, the contrast is reduced by at least 10% of the full dose, preferably at least 20% of the full dose. In some embodiments, implementation of the image reconstruction paradigms generated by the disclosed machine learning methods allows practitioners to reconstruct an equivalent high-resolution contrast-enhanced CT image from a CT image obtained from a patient who was administered at most 80% of the lowest (standard or regulatory approved) concentration of iodinated contrast typically administered in view of the anatomical region to be imaged in an analogous or otherwise healthy counterpart patient. As used herein, an “equivalent” high-resolution contrast-enhanced CT image is intended to include those images having about the same signal to noise ratio and essentially equal diagnostic value.

Alternatively, the contrast dosage administered to the patient can be any dose selected between the foregoing values or within a range defined by upper and lower bounds selected from one of the foregoing values. For example, the reduction in contrast can be between about 1-10%, between about 10-20%, greater than 0% and less than or equal to about 10%, greater than 0% and less than or equal to about 20%, greater than or equal to about 10% and less than or equal to about 20%, at most 10%, or at most 20%. In some embodiments, a low-dose, contrast-enhanced CT image is obtained from a patient having received a contrast dosage calculated to be at least 10%, preferably at least about 20%, more preferably at least about 33% less than a full-dose of contrast.

The reduction in administered contrast agent can make the patient experience more enjoyable or less uncomfortable. For example, a full dose of contrast for Patient A is 150 μg, which is administered via a 3 μg/mL intravenous solution over 50 seconds. Increasing the concentration (and thereby reducing the administration time) can cause nausea or other more serious complications and increasing the rate can be uncomfortable (or painful) for the patient and/or cause the IV port to fail, potentially catastrophically.

By practicing embodiments disclosed herein, Patient A can be administered a low-dose of contrast agent without significantly affecting the resulting CT image quality and/or accuracy (i.e., by generating an equivalent CT image). In an exemplary case, a “low dose” of contrast agent for Patient A is 30 μg (20% of the full dose), which if administered at 5 μg/mL could be administered in six seconds. If administered, for example, at a lower concentration, such as 1 μg/mL, the contrast could be administered to Patient A in 30 seconds—a lower concentration of contrast in less time than the full dose. This can result in a better or less painful experience for the patient while still providing an equivalent contrast-enhanced CT image having the same or about the same diagnostic value.

A deep learning algorithm can be trained using data obtained from a prospective study where patients receive both a low-dose (or an ultra-low dose) of iodinated contrast followed by CT imaging and a routine dose of iodinated contrast followed by CT imaging. The input images for training include those CT images obtained from routine-dosed individuals, and the output images for training include those CT images obtained from low-dose individuals. The ability to reduce iodinated contrast dose provides a major cost savings and reduces risk of adverse events in the patient.

In some embodiments, additional denoising filters and/or denoising convolutional neural networks can be applied to output images.

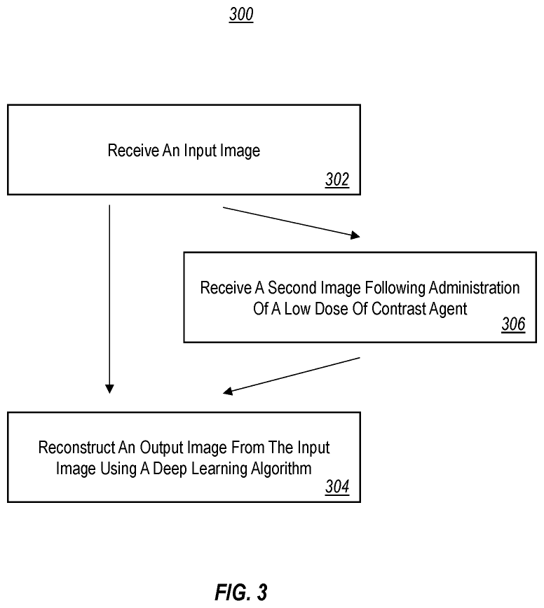

Referring now to FIG. 3, illustrated is a flow chart of an exemplary method 300 for reconstructing an image. The method 300 includes receiving an input image (act 302). The method also includes reconstructing an output image from the input image using a deep learning algorithm (act 304). Act 304 can be implemented to, for example, generate a reconstructed single energy contrast-enhanced CT image from a non-contrast single energy CT image. Alternatively, the deep learning algorithm can reconstruct an output image (act 304) from a single-energy CT input image, the output image being a virtual dual-energy CT image. In some instances, the method 300 can additionally include receiving a second image following administration of a low dose of contrast agent to the patient (act 306). In such an embodiment, the output image is reconstructed from the input image using a deep learning algorithm (act 304) without sacrificing image quality and/or accuracy. It should be appreciated that acts 302, 304, and 306 can be implemented using the systems disclosed and discussed with respect to FIGS. 1 and 2.

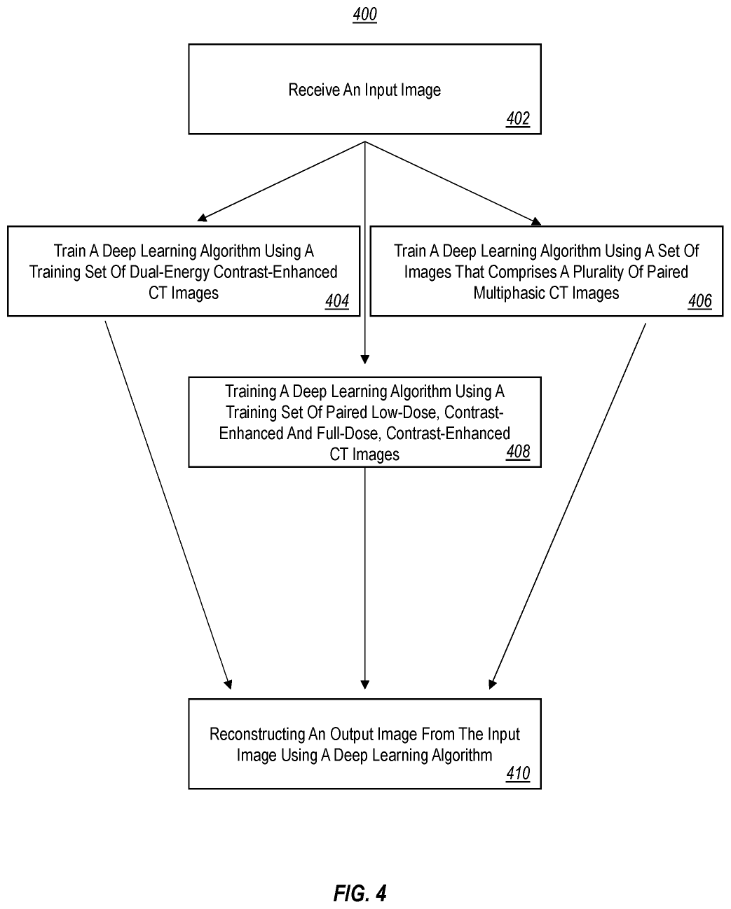

Referring now to FIG. 4, illustrated is a flow chart of an exemplary method 400 for reconstructing an image. The method 400 includes receiving an input image (act 402). The method also includes training a deep learning algorithm using a training set of dual-energy contrast-enhanced CT images (act 404). Act 404 can further include for each dual-energy, contrast-enhanced CT image within the training set, a 70 keV portion of an associated dual-energy, contrast-enhanced CT image is used as a training input CT image and the associated dual-energy, contrast-enhanced CT image is used as a training output CT image.

Additionally, or alternatively, method 400 includes training a deep learning algorithm using a set of images that comprises a plurality of paired multiphasic CT images (act 406). Act 406 can further include each of the paired multiphasic images comprises a nonenhanced CT image and a contrast-enhanced CT image of substantially the same slice from the same patient and/or each of the paired multiphasic images comprises a nonenhanced CT image and a contrast-enhanced CT image of substantially the same slice from the same patient.

Additionally, or alternatively, method 400 includes training a deep learning algorithm using a training set of paired low-dose, contrast-enhanced and full-dose, contrast-enhanced CT images (act 408). Act 408 can further include for each pair of low-dose, contrast-enhanced and full-dose, contrast-enhanced CT images within the training set, the low-dose, contrast-enhanced CT image is used as a training input CT image and the associated full-dose, contrast-enhanced CT image is used as a training output CT image.

Method 400 additionally includes reconstructing an output image from the input image using a deep learning algorithm (act 304).

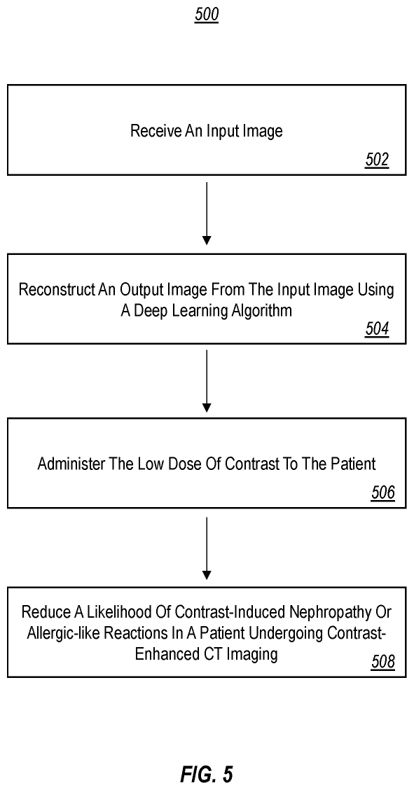

FIG. 5 illustrates yet another flow chart of an exemplary method 500 for reconstructing an image. The method 500 includes receiving an input image (act 502), reconstructing an output image from the input image using a deep learning algorithm (act 504), and administering the low dose of contrast to the patient (act 506). As a result, method 500 includes reducing a likelihood of contrast-induced nephropathy or allergic-like reactions in the patient undergoing contrast-enhanced CT imaging (act 508).

Advantageously, methods such as method 500 illustrated in FIG. 5 additionally provide the unexpected benefit of enabling the reconstruction of a virtual output image equivalent to (e.g., with respect to signal to noise ratio, accuracy, and/or diagnostic value) a contrast-enhanced CT image captured with a normal (i.e., standard) dose of contrast agent. As previously noted, prior efforts at reducing intravenous contrast coupled with known post-processing techniques have been unsuccessful. Embodiments of the present disclosure advantageously provide a solution, enabling the reduction of intravenous contrast agent delivered to a patient during an imaging study. In particular, such methods have proven useful in reducing the concentration of iodinated contrast administered to a patient by as much as 20% without sacrificing image quality and/or accuracy.

### Abbreviated List of Defined Terms

To assist in understanding the scope and content of the foregoing and forthcoming written description and appended claims, a select few terms are defined directly below.

The term “healthcare provider” as used herein generally refers to any licensed and/or trained person prescribing, administering, or overseeing the diagnosis and/or treatment of a patient or who otherwise tends to the wellness of a patient. This term may, when contextually appropriate, include any licensed medical professional, such as a physician (e.g., medical doctor, doctor of osteopathic medicine, etc.), a physician's assistant, a nurse, a phlebotomist, a radiology technician, etc.

The term “patient” generally refers to any animal, for example a mammal, under the care of a healthcare provider, as that term is defined herein, with particular reference to humans under the care of a radiologist, primary care physician, referred specialist, or other relevant medical professional associated with ordering or interpreting CT images. For the purpose of the present application, a “patient” may be interchangeable with an “individual” or “person.” In some embodiments, the individual is a human patient.

The term “physician” as used herein generally refers to a medical doctor, and particularly a specialized medical doctor, such as radiologist. This term may, when contextually appropriate, include any other medical professional, including any licensed medical professional or other healthcare provider.

The term “user” as used herein encompasses any actor operating within a given system. The actor can be, for example, a human actor at a computing system or end terminal. In some embodiments, the user is a machine, such as an application, or components within a system. The term “user” further extends to administrators and does not, unless otherwise specified, differentiate between an actor and an administrator as users. Accordingly, any step performed by a “user” or “administrator” may be performed by either or both a user and/or an administrator. Additionally, or alternatively, any steps performed and/or commands provided by a user may also be performed/provided by an application programmed and/or operated by a user.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

### Computer Systems of the Present Disclosure

It will be appreciated that computer systems are increasingly taking a wide variety of forms. In this description and in the claims, the term “computer system” or “computing system” is defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Although the subject matter described herein is provided in language specific to structural features and/or methodological acts, it is to be understood that the subject matter defined in the appended claims is not necessarily limited to the described features or acts so described. Rather, the described features and acts are disclosed as example forms of implementing the claims.

### CONCLUSION

Unless defined otherwise, all technical and scientific terms used herein have the same meaning as commonly understood by one of ordinary skill in the art to which the present disclosure pertains.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A method for reconstructing an image, comprising:
receiving an input computed tomography (CT) image; and
reconstructing an output CT image from the input CT image using an image reconstruction algorithm generated from a supervised convolutional neural network having one or more parameters of one or more layers of the supervised convolutional neural network informed by received user input.

2. The method of claim 1, wherein the input CT image is a nonenhanced CT image and reconstructing the output CT image comprises reconstructing a virtual contrast-enhanced CT image from the nonenhanced CT image.

3. The method of claim 2, further comprising training the convolutional neural network using a set of images that comprises a plurality of paired multiphasic CT images, wherein each of the paired multiphasic images comprises a nonenhanced CT image and a contrast-enhanced CT image of substantially a same slice from a same patient.

4. The method of claim 1, wherein the input CT image is a contrast-enhanced CT image and reconstructing the output CT image comprises reconstructing a virtual nonenhanced CT image from the contrast-enhanced CT image.

5. The method of claim 4, further comprising training the convolutional neural network using a set of images that comprises a plurality of paired multiphasic CT images, wherein each of the paired multiphasic images comprises a nonenhanced CT image and a contrast-enhanced CT image of substantially a same slice from a same patient.

6. The method of claim 1, wherein the input CT image is a single-energy, contrast-enhanced or unenhanced CT image.

7. The method of claim 6, wherein reconstructing the output CT image comprises reconstructing a virtual dual-energy, contrast-enhanced CT image from the single-energy, contrast-enhanced or unenhanced CT image.

8. The method of claim 7, further comprising training the convolutional neural network using a training set comprising a plurality of dual-energy contrast-enhanced CT images, wherein for each dual-energy, contrast-enhanced CT image within the training set, a 70 keV portion of an associated dual-energy, contrast-enhanced CT image is used as a training input CT image and the associated dual-energy, contrast-enhanced CT image is used as a training output CT image.

9. The method of claim 1, wherein the input CT image is a low-dose, contrast-enhanced CT image.

10. The method of claim 9, wherein the low-dose, contrast-enhanced CT image is obtained from a patient having received a contrast dosage calculated to be at least 10% less than a full-dose of contrast.

11. The method of claim 9, wherein the low-dose, contrast-enhanced CT image is obtained from a patient having received a contrast dosage calculated to be between about 10-20% of a full-dose of contrast.

12. The method of claim 9, wherein the low-dose, contrast-enhanced CT image is obtained from a patient having received a contrast dosage calculated to be at least 10%, preferably at least about 20%, more preferably at least about 33% less than a full-dose of contrast.

13. The method of claim 10, wherein the contrast is intravenous iodinated contrast.

14. The method of claim 13, wherein reconstructing the output image comprises reconstructing a virtual full-dose, contrast-enhanced CT image from the low-dose, contrast-enhanced CT image, the virtual full-dose, contrast-enhanced CT image being reconstructed without sacrificing image quality or accuracy.

15. The method of claim 14, further comprising training the convolutional neural network using a training set of paired low-dose, contrast-enhanced and full-dose, contrast-enhanced CT images, wherein for each pair of low-dose, contrast-enhanced and full-dose, contrast-enhanced CT images within the training set, the low-dose, contrast-enhanced CT image is used as a training input CT image and the associated full-dose, contrast-enhanced CT image is used as a training output CT image.

16. The method of claim 13, further comprising reducing a likelihood of contrast-induced nephropathy or allergic-like reactions in a patient undergoing contrast-enhanced CT imaging, wherein reducing the likelihood of contrast-induced nephropathy or allergic-like reactions in the patient comprises administering the low dose of contrast to the patient prior to or during CT imaging.

17. A computer program product having stored thereon computer-executable instructions that, when executed by one or more processors of a computer system, cause the computer system to reconstruct virtual contrast-enhanced CT images from a patient undergoing nonenhanced CT imaging by performing at least the method of claim 3.

18. A computer program product having stored thereon computer-executable instructions that, when executed by one or more processors of a computer system, cause the computer system to reconstruct nonenhanced CT image data from a patient undergoing contrast-enhanced CT imaging by performing at least the method of claim 5.

19. A computer program product having stored thereon computer-executable instructions that, when executed by one or more processors of a computer system, cause the computer system to reconstruct dual-energy, contrast-enhanced CT image data from a patient undergoing single-energy, contrast-enhanced or nonenhanced CT imaging by performing at least the method of claim 8.

20. A computer system for reconstructing an image, comprising:
one or more processors; and
one or more hardware storage devices having stored thereon computer-executable instructions, when executed by the one or more processors, cause the computer system to perform at least the following:
receive a low-dose, contrast-enhanced computed tomography (CT) image captured from a patient who received a dosage of intravenous iodinated contrast calculated to be at least 10% less than a full-dose of intravenous iodinated contrast; and
reconstruct an output CT image from the low-dose, contrast-enhanced CT image using an image reconstruction algorithm generated from a convolutional neural network, the output CT image comprising a virtual full-dose, contrast-enhanced CT image,
wherein the convolutional neural network is trained using a training set of paired low-dose, contrast-enhanced and full-dose, contrast-enhanced CT images such that for each pair of low-dose, contrast-enhanced and full-dose, contrast-enhanced CT images within the training set, the low-dose, contrast-enhanced CT image is used as a training input CT image and the associated full-dose, contrast-enhanced CT image is used as a training output CT image.
